(19) United States
(12) Plant Patent Application Publication
(10) Pub. No.: US 2003/0121087 P1
(43) Pub. Date: Jun. 26, 2003

(54) Chrysanthemum plant named 'Esperanto'

(76) Inventor: Richard Hammond, Gran Canaria (ES)

Correspondence Address: Mark K. Dickson, HOWREY SIMON ARNOLD & WHITE, LLP, 301 Ravenswood Avenue, Menlo Park, CA, 94025, US

(21) Appl. No.: 10/029,888

(22) Filed: Dec. 21, 2001

(51) Int. Cl. A01H 5/00

(52) U.S. Cl. PLT/286

(57) ABSTRACT

A new variety of Chrysanthemum plant named ‘Esperanto,’ having a good uniform canopy of bright purple/white bi-colored single flowers with a yellow/green disc. The new variety has medium vigor with a free branching and uniform spreading habit and good foliage presentation.

### LATIN NAME OF THE GENUS AND SPECIES

Botanical classification: Dendranthema grandiflora.

### VARIETY DENOMINATION

The new Chrysanthemum variety denomination is ‘Esperanto.’

### BACKGROUND OF THE INVENTION

The present invention comprises a new and distinct cultivar of Chrysanthemum botanically known as Dendranthema grandiflora, and referred to by the cultivar name ‘Esperanto.’

‘Esperanto,” originated from a cross made in a controlled breeding program at Valsequilla, Gran Canaria, Spain. The female parent of the new variety is an undistributed, unpatented seedling identified as 715-20 and the male parent is the variety known as undistributed, unpatented seedling identified as R610-30. The new variety ‘Esperanto’ has been asexually reproduced by vegetative cuttings in Chichester, West Sussex, United Kingdom, and the distinguishing characteristics are retained through successive generations of asexual reproduction.

### BRIEF SUMMARY OF THE INVENTION

‘Esperanto’ is a pot type of Chrysanthemum plant variety having single type flowers formed by purple/white stripped petals, the flowers forming a flat capitulum.

### Comparison with Parent

Plants of the new Chrysanthemum variety ‘Esperanto’ are similar to the parent cultivar 715-20 in plant habitat and growth rate. In side-by-side comparisons in Chichester, West Sussex, United Kingdom, under commercial practice, plants of the new Chrysanthemum variety ‘Esperanto’ differed from plants of the parent cultivar 715-20 in the following characteristics.

1. The new variety ‘Esperanto’ produces white with pink flowers whereas the parent cultivar 715-20 produces white single flowers.

2. Plants of the new variety ‘Esperanto’ have larger and shorter inflorescence than plants of the parent cultivar 715-20.

### Comparison with Other Varieties

There are no known comparable varieties.

### BRIEF DESCRIPTION OF ILLUSTRATIONS

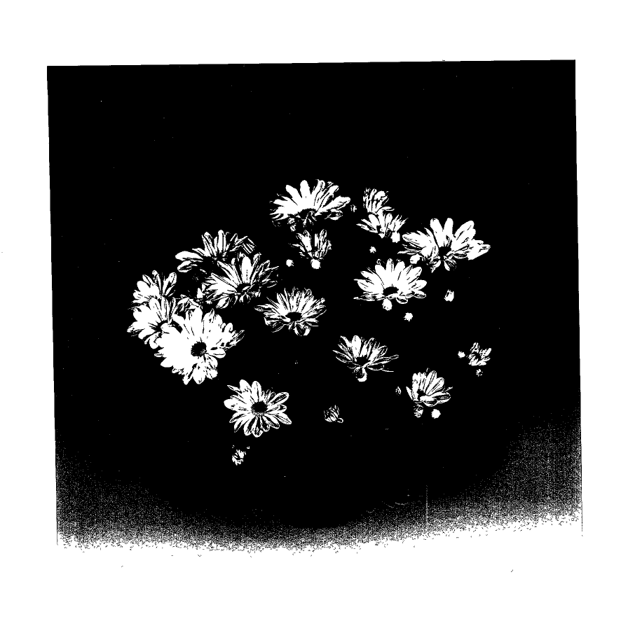

Typical specimens of the plant and flowers for the new Chrysanthemum variety ‘Esperanto’ are shown in the accompanying photograph. The colors shown are as true as possible within the usual limits of this kind of illustration. FIG. 1 is a whole plant view of the new variety ‘Esperanto’ grown in a pot. The plant shown in the illustration is 53 days from the commencement of short days.

### DETAILED BOTANICAL DESCRIPTION

The following description of the new Chrysanthemum variety ‘Esperanto’ is of plants grown in a greenhouse in Chichester, West Sussex, United Kingdom, in the month of October. The cultivar has not been observed under all possible environmental conditions. The phenotype may vary significantly with variations in the environment such as temperature, length of day and light intensity, without any variance in genotype. The commercial classification of the new variety is a pot Chrysanthemum.

Plants of the new variety have been grown successfully under temperature conditions averaging about 18° C. at night and about 24° C. to 25° C. during the day under light conditions of about 5,000 to 6,000 foot candles. The plants respond well to the use of growth retardant, such as B9. To produce a commercial product the plants may be pinched once with the center bud removed. The typical container size for commercial growth is 14 cm. It has been observed that the shelf life of the new variety is about 24 days with a response time of about 7½ weeks. The new variety is suitable for growth in a temperature range of 17° C. to 28° C. during short days.

The following description is with respect to a plant produced as a pot plant In the description of this new Chrysanthemum variety, color values have been taken from The Royal Horticultural Society Colour Chart (R.H.S.C.C.).

### Plant

Plant type: Pot

Habit: Upright and Branching

Height: Medium

Width: Near 35 cm

Branching characteristics (describe): Free and spreading

Length of Lateral Branches: 6 cm-11 cm

Number of Breaks from Pinch: 3 to 5

Stem Color: 137A

Response Time: 53 days

Vigor: Medium

Shelf Life: Near 24 days

Disease (susceptibility or resistance observed): No observations

Pest (susceptibility or resistance observed): No observations

Growth retardant type and treatment: 3 applications of 2.5 grams/litre at 2 days, 21 days and 28 days after sticking of unrooted cuttings The plants were grown for 2 weeks in Long Day conditions (20 hours of light) and then transferred to Short Day conditions (13 hours of dark).

Propagation:

Type.—Vegetative propagation via stem cuttings.

Time to rooting.—12-14 days with soil temperatures of 18° C.

Rooting habit.—Fine and fibrous.

### Foliage

Number of Leaves Per Lateral Branch: 8-11

Compound or single: Single

Arrangement of Leaves: Alternate

Shape of leaf.—Typically 5 lobed ovate.

Size of leaf.—Width (cm): near 3.5 cm-7 cm Length (cm): near 5 cm-10 cm.

Leaf apex.—Acute.

Base.—Obtuse.

Attachment.—Petioled.

Aspect.—Slightly undulating.

Margin.—Palmately lobed.

Surface characteristics.—Top: Slightly pubescent Bottom: Pubescent.

Petiole:

Color.—near 137B.

Length.—near 1.5 cm.

Venation (describe): Net prominent midvein at underside

Color.—upper side: near 146D under side: near 146D.

Color:

Mature Leaf, upper side.—near 137A; under side: near 138A.

Young Leaf, upper side.—near 137B; under side: near 138B.

### Flower

Flower appearance: Circular slightly cupped

Flower type: Single

Flower form: Slightly cupped

Flower shape: Round

Flowering habit: Cyme

Number of blossoms per branch: 3-5

Inflorescence form: Pyramidal

Depth of fully expanded blossoms.—near 2 cm.

Diameter of fully expanded blossoms.—6 cm-8 cm.

Sepals:

Number.—28-30.

Color.—near 136C.

Length.—0.5 cm-0.9 cm.

Width.—0.2 cm-0.4 cm.

Texture/Appearance.—Pubescent.

Peduncle: longest length is on average 10 cm, shortest length is on average 2.5 cm:

Length.—between 2.5 and 10 cm.

Color.—near 136C.

Surface.—Pubescent.

Habit.—Slightly curving.

Strength.—Medium.

Pedicel:

Length.—terminal: 2 cm-4.5 cm; lateral: 2.5 cm-7 cm.

Color.—near 138B.

Surface.—Pubescent.

Habit.—Slightly curving.

Strength.—Medium.

Ray Florets:

Form/Shape.—Straight.

Texture/Appearance.—Matte.

Number per flower.—25-30.

Length.—3 cm-3.5 cm Width: 0.8 cm-1.2 cm.

Apex.—Rounded.

Base.—Tapered.

Margin.—Entire.

Disc florets:

Form/Shape.—Cylindrical.

Texture/Appearance.—Shiny.

Number per flower.—170-250.

Length.—near 0.3 cm-0.6 cm Width: <0.1 cm.

Diameter of disc.—1.2 cm-1.5 cm.

Fragrance: Faint

Flower Bud (at onset of color):

Length.—near 1.5 cm.

Diameter.—near 1 cm.

Form/shape.—Globular.

General flower color:

1. Ray florets, upper side.—Immature: near 73A; lattice near 155B Mature: near 73B; lattice near 155B Older/Fading: near 73C; lattice near 155B.

2. Ray florets, under side.—Immature: 73D; lattice near 155B Mature: 73D; lattice near 155B Older/Fading: 73D; lattice near 155B.

3. Disc florets.—Immature: near 143B Mature: near 154B Older/Fading: near 143A.

4. Bud.—near 138B Flower progression with age: Less color differential as flowers mature — more fading of colors. No significant flower form change.

### Reproductive Organs

Ray Florets: Pistillate

Pistil member.—

Stigma color.—near 6C.

Stigma shape.—Forked.

Style color.—near 6C.

Style length.—near 0.4 cm.

Disc Florets: Pistillate

Stigma color.—near 6C.

Stigma shape.—Forked.

Style color.—near 6C.

Style length.—near 0.2 cm.

Staminate.—

Stamen number.—None observed.

Fruit and Seeds: None

What is claimed is:

1. A new and distinct variety of Chrysanthemum plant, substantially as described and illustrated herein.
